(19) United States
(12) Patent Application Publication
(10) Pub. No.: US 2016/0310061 A1
(43) Pub. Date: Oct. 27, 2016

(54) Method of monitoring of the onset of labor in a mammal

(71) Applicants: Jean Rebecca Campero, Gilroy, CA (US); Catherine Louise Register, Los Gatos, CA (US)

(72) Inventors: Jean Rebecca Campero, Gilroy, CA (US); Catherine Louise Register, Los Gatos, CA (US)

(21) Appl. No.: 14/693,875

(22) Filed: Apr. 23, 2015

(51) Int. Cl. A61B 5/00; A61D 17/00; A61B 5/145

(52) U.S. Cl. CPC A61B 5/4343; A61B 5/14507; A61B 5/14539; A61B 5/14546; A61B 5/6831; A61B 5/746; A61D 17/00

(57) ABSTRACT

A method of monitoring of the onset of labor in a mammal such as a dog, livestock, horse, exotic animal, cat, or endangered animal through the detection of amniotic fluid is described. Detection of the onset of labor is desired so that a caretaker, veterinary professional, owner, or other personnel can provide a timely response

### BACKGROUND

The present invention relates to a method of detecting when mammals such as dogs, livestock, horses, exotic animals, cats, or endangered animals are about to enter labor through the detection of amniotic fluid.

During the pregnancy of mammals, the baby is surrounded and cushioned by the amniotic sac which is a fluid-filled membrane. Typically, at the beginning of labor, the amniotic sac will rupture releasing the amniotic fluid. Mammals can be in pre-labor for a considerable amount of time and the timely response, management and treatment of the animal may require constant supervision of a caretaker, veterinary professional, owner, or other personnel to identify when the animal is entering labor.

Uterine monitoring devices based on a tocodynometer to sense uterine irritability and uterine contractions have been popular for decades on humans and have just been recently applied to other mammals (U.S. Pat. No. 5,400,799). These devices can be used to detect and predict when an animal has or may be entering labor. These devices are expensive and are not readily available for the monitoring of animals outside of a veterinary clinic.

Amniotic fluid can contain carbohydrates, proteins, peptides, lipids, lactate, pyruvate, electrolytes, enzymes, and hormones. The specific types of carbohydrates, proteins, peptides, lipids, lactate, pyruvate, electrolytes, enzymes, and hormones present in the amniotic fluid depends on the species of mammal. The present invention considers the presence of amniotic fluid to be the detection or measurement of one or more of the following: water, urine, pH, carbohydrates, proteins, peptides, lipids, lactate, pyruvate, electrolytes, enzymes, and hormones.

The present invention provides a method to detect the presence of amniotic fluid and to alert the caretaker, veterinary professional, owner, or other personnel that the animal under monitoring has entered labor. The method described by the present invention utilizes commonly available technology components; thereby, providing for a low cost monitoring system that can be used in locations within or outside of a veterinary clinic.

### SUMMARY

Monitoring of the onset of labor in a mammal such as a dog, livestock, horse, exotic animal, cat, or endangered animal is desired so that a caretaker, veterinary professional, owner, or other personnel can provide a timely response. It is desirable to have the detection method be able to be performed outside of a clinic and be easy and reliable to use.

### BRIEF DESCRIPTION OF THE DRAWINGS

FIG. 1 shows the application of a typical amniotic fluid measurement device.

FIG. 2. shows the components that make up an amniotic fluid measurement device.

FIG. 3. is a flow diagram of the steps performed during monitoring.

### DETAILED DESCRIPTION

In the following description, reference is made to the accompanying drawings which form a part hereof and which illustrate several embodiments. It is understood that other embodiments may be utilized and structural and operational changes may be made without departing from the scope of the descriptions provided.

### INTRODUCTION

The present invention involves the use of an electronic sensor and monitor to detect amniotic fluid from a mammal such as a dog, livestock, horse, exotic animal or endangered animal. Once the amniotic fluid has been detected the owner, caretaker, veterinary professional or other personnel can be alerted by the monitor so that proper care can be given to the animal. The present invention considers the presence of amniotic fluid to be the detection of one or more of the following conditions or components that are known to be present in amniotic fluid: presence of fluid which could be water, secretions, or urine; pH measurement of the fluid; presence of carbohydrates; presence of proteins; presence of peptides; presence of lipids; presence of lactate; presence of pyruvate; presence of electrolytes; presence of enzymes; or the presence of hormones known to be present in the amniotic fluid. The sensor designs needed for detection of some of these components are well know in the prior art.

### MEASUREMENT DEVICE

Preferred embodiments and applications will now be described. Other embodiments may be realized and changes may be made to the disclosed embodiments without departing from the spirit or scope of the inventions described herein. Although the preferred embodiments disclosed herein have been particularly described as applied to the method of monitoring of the onset of labor in a mammal such as a dog, livestock, horse, exotic animal, cat, or endangered animal through the detection of amniotic fluid, it should be readily apparent that the invention may be applied to any application having the same or similar problems.

In the following description, a reference is made to the accompanying drawings which form a part hereof and which illustrate several embodiments. It is understood that other embodiments may be utilized and structural, configurational, and/or operational changes may be made without departing from the scope of the descriptions provided.

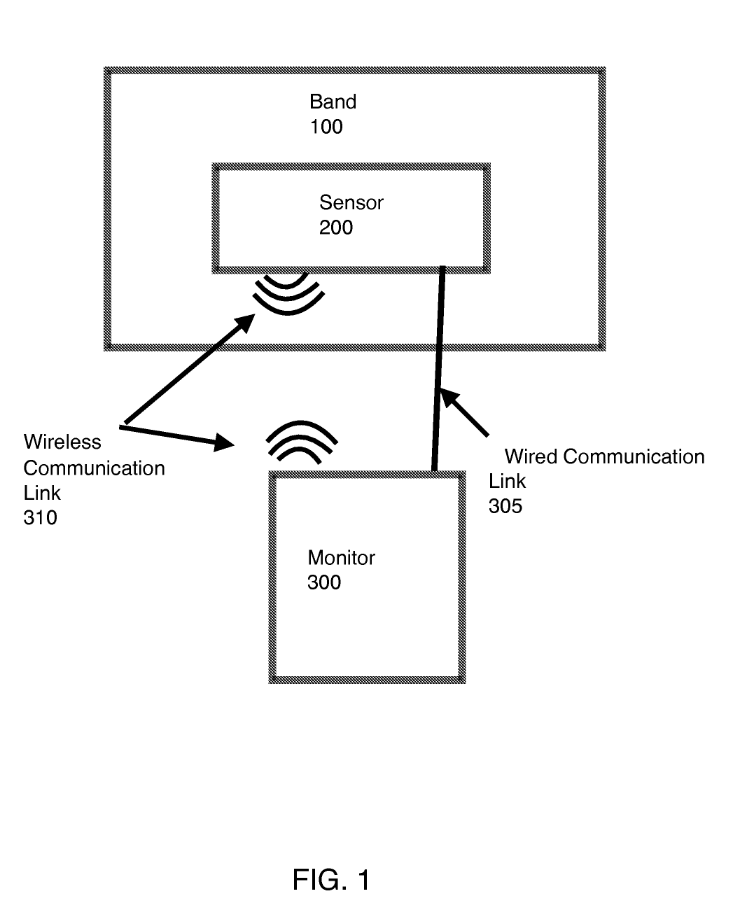

FIG. 1. Shows the application of a typical amniotic fluid measurement device for the monitoring of a mammal. The amniotic fluid sensor 200 is attached to a band 100 that can be used for the placement of the sensor onto the mammal. The sensor 200 can also be applied through other means such as adhesive tape. The sensor 200 is to be positioned so that the amniotic fluid will contact the sensor when the amniotic membrane ruptures. The band 100 can be a band made from elastic material or it could be a diaper or a set of bloomers. The monitor 300 could be connected to the sensor 200 through a wired connection 305 or it could use a wireless communications link 310 using a proprietary wireless communication protocol or an industry standard communication protocol such as 802.11, BlueTooth or other industry standard communications method. The monitor 300 is used to alert the caretaker, veterinary professional, caretaker, or other personnel to the detection of the amniotic fluid through a visual, audible, or other altering mechanism. The monitor 300 can be a hardware device or it could be a commodity Andrioid, Windows, iOS, or Linux type device running an application that is monitoring the sensor 200.

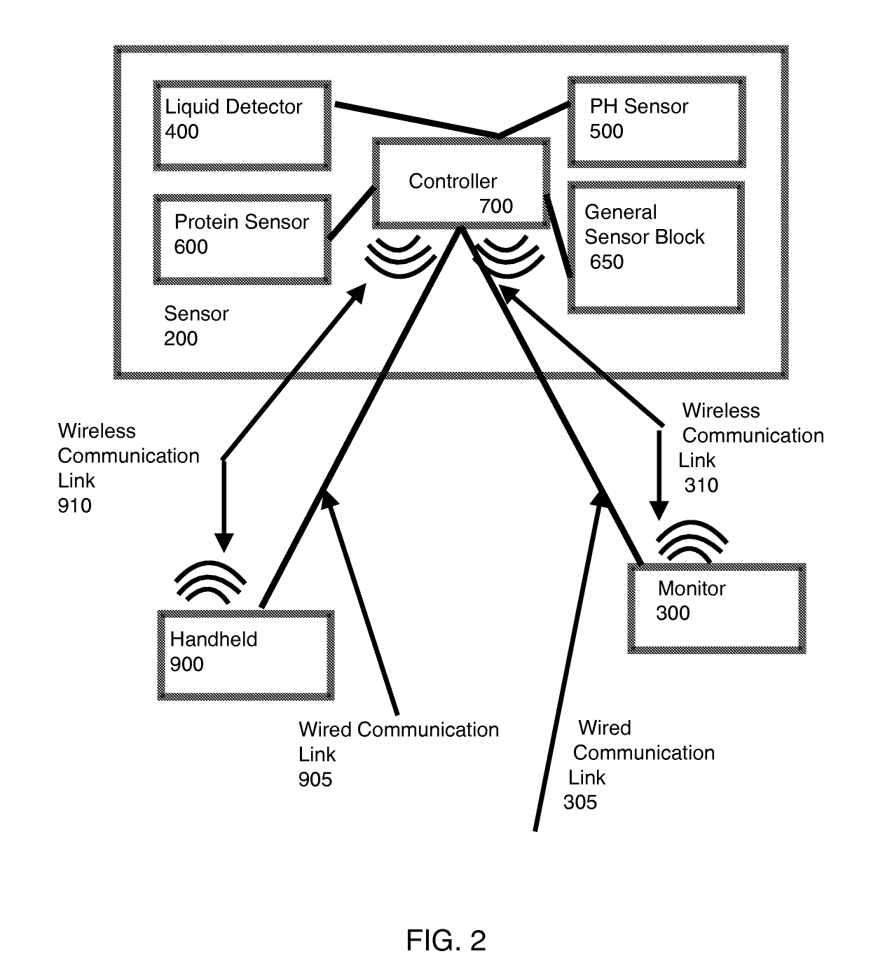

FIG. 2. Shows the major functional blocks of the sensor 200. The sensor is composed of one or more functional blocks that can detect liquid 400, measure PH 500, detect proteins 600, or a general purpose detector sensor or a sensor array 650 that can detect one or more of the following compounds: carbohydrates, proteins, peptides, lipids, lactate, pyruvate, electrolytes, enzymes, and hormones. The sensor elements are controlled and monitored by a controller 700. Controller 700 measures the sensors 400, 500, 600, 650 outputs and determines if amniotic fluid is present and if amniotic fluid is detected then the controller can send the sensor reading values to the remote monitor 300 for analysis and alerting. The controller 700 can also make the determination that amniotic fluid is present based on the sensor readings and can send the detection status of amniotic fluid to the remote sensor 300 for alerting. The remote monitor 300 will alert the caretaker, veterinary professional, caretaker, or other personal to the detection of the amniotic fluid through a visual, audible, or other altering mechanism. The remote monitor 300 can make the decision that amniotic fluid has been detected based on the raw data obtained from the sensor controller 700 or the remote controller 300 can be told to alert by the sensor controller 700.

In one embodiment, the controller 700 will send an alert to the monitor which could be a commodity based handheld device 900 such as a smart phone, tablet, or other device through an industry standard communications protocol or through a proprietary protocol. The connection from the handheld 900 to the controller 700 could also be through a wired connection 905 or wireless connection 910. In these embodiments, the handheld device 900 would alert the caregiver. The handheld device 900 could also display the status of each sensor monitored by the controller 700 and it could also display the analytical readings of each of the sensors. For example, the handheld 900 could display the pH that is being measured or it could display the concentration of proteins that are being detected. These same use cases could also be displayed by the remote monitor 300.

The remote monitor 300, depending on the implementation, can display the same sort of information as the handheld 900 application if it contains a display mechanism or it could be just a simple device that can only alert the caregiver that the amniotic fluid has been detected. In both embodiments, the monitor 300 could receive the information from the controller 700 via either a wireless link or through a wired connection. The wireless 310 or wired 305 connection can be any of the well established protocols known in the art or it could be a proprietary protocol.

The monitor 300 can receive data from the sensor 200, can receive alerts from the sensor 200, or it can receive a combination of data and alerts. The monitor 300 can contain components that allow display of the data transmitted by the sensor 200 or it can provide just alerting functionality or a combination of both.

The monitor 300 could also send the data and alerts to the handheld 900 directly through a wired or wireless connection rather than the 900 being connected to the controller 700.

The fluid sensor 400 alerts to the presence of any liquid and can therefore cause false alerts as the presence of water, urine or other liquids could cause the sensor to activate. To improve the accuracy of the system, the fluid sensor 400 can be used in combination with another sensor. The pH sensor 500 can be used to measure the pH of the fluid that has come in contact with the sensor 200. The pH threshold could be adjusted to the specific pH levels of the mammals amniotic fluid. The pH sensor 500 can be used alone or in combination with one of the other sensors. The amniotic fluid for the mammal may have a pH range that is outside the range of water, urine and other liquids thereby improving the detection accuracy of the sensor 200. The general sensor block 650 can be used alone or in combination with the other sensor elements. The amniotic fluid can contain carbohydrates, proteins, peptides, lipids, lactate, pyruvate, electrolytes, enzymes, and hormones. The general sensor block 650 could contain elements that can specifically detect one or more of the carbohydrates, proteins, peptides, lipids, lactate, pyruvate, electrolytes, enzymes, or hormones that are contained within the mammals amniotic fluid. The general sensor block 650 can significantly improve the false detection rate as the detected presence of these components is not likely to be from a source other than the amniotic fluid.

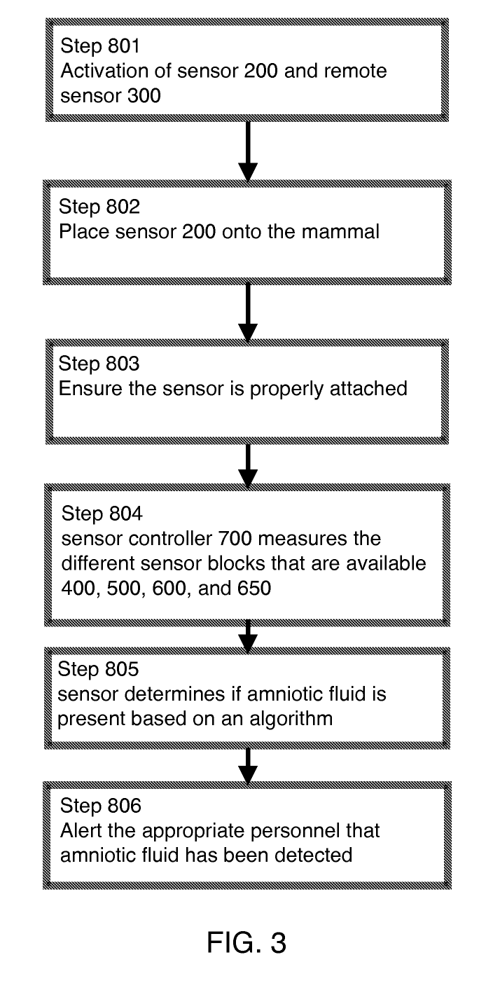

FIG. 3. is an example flow diagram of the steps performed during monitoring. It is understood that this is one series of steps for illustration that could be performed but as taught in the specification many other sequences of steps can be used during monitoring; these steps could be static or they could also be dynamic and change with respect to the device operation and configuration. The first step 801 is the activation of the sensor 200 and remote sensor 300. The second step 802 is to place the sensor 200 onto the mammal in a position where it will come in contact with the amniotic fluid when the amniotic membrane ruptures. Step 803 is to ensure that the sensor 200 is properly attached. In Step 804, the sensor controller 700 measures the different sensor blocks that are available 400, 500, 600, and 650. In step 805, the sensor determines if amniotic fluid is present based on an algorithm. The algorithm is based on the sensors that are available and can be based on a single sensor reading or a combination of sensor reading values. The most basic algorithm monitors sensor 400 for the presence of any fluid and sends an alert to the remote monitor 300. A more advanced algorithm can combine the fluid sensor 400 with another sensor such as the pH sensor 500 or the general sensor block 600. In this algorithm, the controller 700 will check the status of sensors 500 or 600 if the presence of liquid is detected by fluid sensor 400 and it will only alert the remote monitor 300 if the sensor 500 is within the proper pH range or sensor 600 has detected one of the carbohydrates, proteins, peptides, lipids, lactate, pyruvate, electrolytes, enzymes, or hormones known to be present in the amniotic fluid; the algorithm could also operate whether or not the presence of liquid is detected by fluid sensor 400. The sensor controller 700 can activate the remote controller 300 if it detects the presence of a fluid in the proper pH range independently of any other sensor readings. The sensor controller can also alert the remote monitor 300 if the general sensor 600 detects the presence of any one of the following carbohydrates, proteins, peptides, lipids, lactate, pyruvate, electrolytes, enzymes, or hormones known to be present in the amniotic fluid. In step 806, the monitor 300 or the handheld 900 or combination thereof will alert the appropriate personnel that the presence of the amniotic fluid has been detected.

The foregoing description of various embodiments has been presented for the purposes of illustration. It is not intended to be exhaustive or to limit to the precise form disclosed. Many modifications and variations are possible in light of the above teaching Those skilled in the art will recognize improvements and modifications to the preferred embodiments of the present invention. All such improvements and modifications are considered within the scope of the concepts disclosed herein and the claims that follow.

In another embodiment, the sensor 200 and the monitor 300 can be combined into a single unit.

What is claimed is:

1. A method of detecting that a mammal is entering labor through the detection of amniotic fluid comprising the steps of:
positioning a sensor on the mammal where it will be contacted by amniotic fluid when the amniotic sac membrane ruptures and
monitoring the sensor for the presence of amniotic fluid and
generating an alert when the presence of amniotic fluid is detected.

2. The method of claim 1 wherein the step of monitoring the sensor for the presence of amniotic fluid to be the detection of one or more of the following conditions or components that are known to be present in amniotic fluid: presence of fluid which could be water, secretions, or urine; pH measurement of the fluid; presence of carbohydrates; presence of proteins; presence of peptides; presence of lipids; presence of lactate; presence of pyruvate; presence of electrolytes; presence of enzymes; or the presence of hormones known to be present in the amniotic fluid.

3. The method of claim 1 wherein the step of generating an alert when the presence of amniotic fluid is detected includes the step of sending the signal to a remote controller for alerting

4. The method of claim 1 wherein the step of generating an alert includes the step of generating one or more audible alert, visual alert, or vibratory alert.

5. The method of claim 1 wherein the mammal is a dog, livestock, horse, exotic animal, cat, or endangered animal.

6. A method of detecting that a plurality of mammals is entering labor through the detection of amniotic fluid from each of the plurality of mammals comprising the steps of:
positioning a sensor on each of the plurality of mammals where it will
be contacted by amniotic fluid from each of the plurality of mammals when the amniotic sac membrane ruptures and
monitoring the plurality of sensors for the presence of amniotic fluid and
generating an alert when the presence of amniotic fluid is detected.
